(19) United States
(12) Patent Application Publication
(10) Pub. No.: US 2021/0045304 A1
(43) Pub. Date: Feb. 18, 2021

(54) SCALE-UP EVALUATION METHODS FOR ALGAL BIOMASS CULTIVATION

(71) Applicant: ExxonMobil Research and Engineering Company, Annandale, NJ (US)

(72) Inventors: Patrick L. Hanks, Bridgewater, NJ (US); William J. Novak, Bedminster, NJ (US); Everett J. O'Neal, Asbury, NJ (US); Louis R. Brown, La Jolla, CA (US)

(21) Appl. No.: 16/891,125

(22) Filed: Jun. 3, 2020

Related U.S. Application Data
(60) Provisional application No. 62/887,926, filed on Aug. 16, 2019.

(51) Int. Cl. A01G 33/00; C12M 1/00; C12M 1/34; C12N 1/12

(52) U.S. Cl. CPC A01G 33/00; C12M 23/18; A01G 24/10; C12N 1/12; C12M 41/42

(57) ABSTRACT

Scale-up evaluation methods for algal biomass cultivation are provided, and more particularly, simultaneous scale-up evaluation methods for outdoor algal biomass cultivation are provided. The methods control various process variables to isolate the impact of scaling algal biomass cultivation, thereby permitting enhanced scale-up operation design to optimize the quality and quantity of algal biomass.

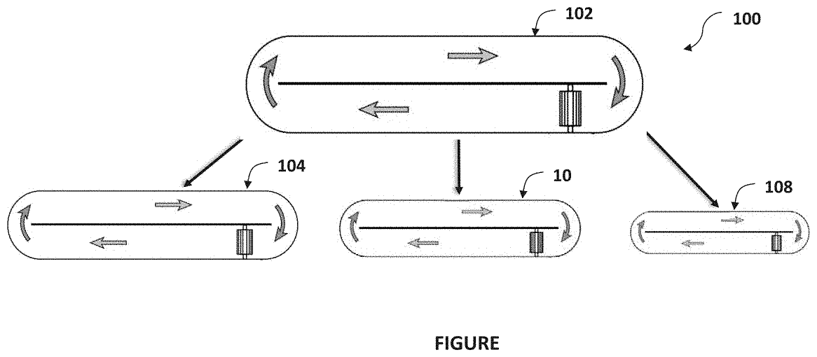

### CROSS-REFERENCE TO RELATED APPLICATIONS

This application claims the benefit of U.S. Provisional Application No. 62/887,926 filed Aug. 16, 2019, which is herein incorporated by reference in its entirety.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources.

Algae are photoautotrophic organisms, or organisms that can survive, grow, and reproduce with energy derived entirely from the sun through the process of photosynthesis. Photosynthesis is essentially a carbon recycling process through which inorganic CO₂ combines with solar energy, other nutrients, and cellular biochemical processes to output gaseous oxygen and to synthesize carbohydrates and other compounds critical to the life of the algae.

To produce algal biomass in outdoor environments, algae is generally grown in a water slurry using one or more open pond systems, which are typically oval in shape (e.g., pill-shaped) and referred to as “raceway ponds.” The water slurry comprises selected nutrients and the pond system circulates the algae in the water slurry to ensure adequate exposure to solar energy, thereby promoting the growth of algal biomass. Various processing methods separate the algal biomass and extract lipids therefrom for the production of fuel and other oil-based products. The remaining wastewater and biomass residue can be recycled or otherwise used in a variety of sustainable applications. For example, the wastewater can form some or all of a subsequent water slurry and the biomass residue can be used as animal feed.

Because the processing of algal biomass produces valuable commodities, including sustainable biofuels, large-scale cultivation of algae is desirable. Various engineering and ecological complications may be associated with scaling an algae cultivation process, such as accounting for light-path distance, hydrodynamic flow pattern, environmental conditions, and others to ensure adequate growth conditions. As such, various volumetrically sized open pond systems are not merely interchangeable.

To date the largest pond systems are upwards of one to two acres in area. However, to compete with merely U.S. diesel demand, which is on the order of about 3 million barrels per day (bpd), a single algae biofuels facility producing diesel would likely need to produce at least 10,000 bpd, or even more (e.g., 20,000 bpd), to be viable, which is on par with current refinery facilities producing petroleum products. Accordingly, the total area of a pond system for true commercial algae biomass production would need to be extremely large, requiring ponds covering hundreds, or even thousands, of acres of total surface area. However, no methodology for determining optimal pond sizes is currently available to those of skill in the art. For example, an optimal pond system may include a single large pond or multiple smaller or intermediate sized ponds—and no prior methodology exists for making this determination. The present disclosure provides a methodology for determining an optimal pond size for algal biomass production, accounting for aforementioned engineering and ecological complications.

### SUMMARY OF THE INVENTION

The present disclosure is related to scale-up evaluation methods for algal biomass cultivation, and more particularly, to simultaneous scale-up evaluation methods for outdoor algal biomass cultivation.

In some embodiments, a method includes mixing an algae water slurry in a first cultivation pond, transferring the algae water slurry into at least two additional cultivation ponds, the at least two additional cultivation ponds having different volumetric sizes and each having a volumetric size less than the first cultivation pond, and cultivating the algae water slurry in the at least two additional cultivation ponds for an equivalent predetermined period of time, and thereby growing an algal biomass. A quality and quantity of the algal biomass may then be evaluated to determine influence of scaling.

### BRIEF DESCRIPTION OF THE DRAWINGS

The FIGURE is included to illustrate certain aspects of the present disclosure, and should not be viewed as exclusive embodiments. The subject matter disclosed is capable of considerable modifications, alterations, combinations, and equivalents in form and function, without departing from the scope of this disclosure.

The FIGURE is a schematic flowchart of a process for scale-up evaluation of algal biomass cultivation, according to one or more embodiments of the present disclosure.

### DETAILED DESCRIPTION

The present disclosure is related to scale-up evaluation methods for algal biomass cultivation, and more particularly, to simultaneous scale-up evaluation methods for outdoor algal biomass cultivation.

Biofuel production from cultivated algae slurries offers sustainable energy solutions to reduce reliance on fossil fuels and reduce greenhouse gas emissions. To accomplish substantial economic, environmental, and societal impact, algae must be cultivated in large-scale systems. Such large-scale cultivation systems further allow algae-derived fuels to become more cost-effective and more widely available to the public. Once an algae cultivation facility has designed and operated an algae cultivation regime that achieves desired results on a small scale, the facility must scale-up its cultivation processes with the objective of enlarging production quantities without compromising productivity or quality. Successful scale-up is vital to the commercial viability of an algae cultivation facility, in terms of at least both operational cost control and algal product quantity. Moreover, successful scale-up is vital to ensure that a facility is achieving desirable production rates (e.g., those that can enable commercial viability).

As used herein, the term “algae slurry,” and grammatical variants thereof, refers to a flowable, liquid comprising at least water, algae cells, and algae nutrient media, discussed in further detail hereinbelow.

Traditional scale-up methods involve initially cultivating a concentrated algae stock solution and aliquoting an amount of the stock solution into a plurality of cultivation raceway ponds of different volumetric sizes to achieve similar algae concentrations, each pond comprising the contents of an algae slurry except for the algae cells. Raceway ponds are shallow, artificial ponds having single or multiple closed-loop, oval-shaped (e.g., pill-shaped) recirculation channels. Paddlewheels and/or other suitable powered mechanical devices (e.g., airlift pumps, mixing boards, and the like) enable circulation and prevent sedimentation of the algae cells contained therein. Raceway ponds are typically designed to circulate an algae slurry at a depth of no greater than about 12 inches (in.) to facilitate sufficient sunlight penetration needed for algae growth. Other shaped cultivation recirculation ponds may be equally applicable to the present disclosure and embodiments described herein, and collectively referred to herein as “cultivation ponds.”

Using traditional scale-up methods, each of the algae slurries in the plurality of cultivation ponds are recirculated at a velocity sufficient to prevent algae sedimentation and are allowed to cultivate for a particular period of time. Evaluation of the resultant algal biomass informs scale-up design. However, such traditional methods fail to control for a variety of process variables that influence the growth and quality of an algae slurry. Examples of weather and biological process variables include, but are not limited to, ambient temperature; photosynthetically active radiation (PAR) exposure; humidity; wind speed and shear; light penetration, salinity, dissolved oxygen, dissolved carbon dioxide, pH, and nutrient media contents of an algae slurry; algae density; algae stress (e.g., based on Fv/Fm chlorophyll fluorescence measuring parameter); and the like; and combinations thereof. For example, water fill time varies for each pond size, thereby influencing the dissolved oxygen in the water prior to inclusion of the algae aliquot. Additionally, mixing at a velocity merely to prevent sedimentation results in different power per unit volume across the different sized ponds, and thus different mixing and agitation across the ponds.

Failure to control for such process variables results in an imprecise comparison of the resultant algal biomass obtained from the plurality of different sized cultivation ponds, and therefore results in imprecise information for scale-up design. That is, the influence of the scale-up operation itself on the quality and quantity of the resultant biomass is not isolated, but contaminated with other non-scale-up, process variables.

The present disclosure provides methods for controlling process variables to isolate the impact of scale-up, thereby improving information available for the design of a scale-up operation. The methods described herein permit simultaneous study on the impact of scale-up that minimizes the differences between initial and temporal (e.g., weather) process variables.

Examples of scale-up process variables encompass the hydrodynamics of the pond cultivation system and include, but are not limited to, the size of the pond, pond wall drag (friction) between the algae slurry and the pond walls, pond bottom drag (friction) between the algae slurry and the pond bottom, thermal conductive exchange between the algae slurry and the pond wall/bottom, and the like, and combinations thereof. For example, algae slurries in smaller ponds experience greater power input per unit volume as compared to algae slurries in larger ponds because they circulate and encounter paddlewheels or other recirculation devices more frequently over a similar time period. This can result in different light-dark exposure times for the algae between the different sized ponds, which can affect the growth of algal biomass and influence the design of a scale-up operation. The methods of the present disclosure may, for example, be used to determine the minimal sized cultivation pond that is required before such light-dark exposure time differences are no longer apparent or within the noise of associated analytical procedures (e.g., procedures such as measuring ash free dry weight, total organic carbon, plant stress (Fv/Fm), and the like, and any combination thereof associated with the growth of algal biomass).

The present disclosure simultaneously seeds a plurality of different sized cultivation ponds with identical algae slurries to control process variables and determine the impact of scaling. At least three cultivation ponds of different volumetric sizes ranging from a largest size to a smallest size are required according to the methods described herein. The area of the cultivation ponds may range, for example, from about 2 square meters (m²) to about 10,000 m², or larger, encompassing any value and subset therebetween. For example, representative areas of suitable cultivation ponds may include about 2 m², about 75 m², about 400 m², about 4000 m², about 6000 m², about 8000 m², and about 2.5 m².

The largest cultivation pond serves as a vast mixing vessel for the preparation of an algae slurry to normalize various process parameters related to the algae slurry, as described herein. Subsequently, the algae slurry is transferred into at least two smaller, otherwise empty cultivation ponds for examination of scale effects. Each of the at least two smaller cultivation ponds are designed to hold a volume of the prepared algae slurry at a depth of no greater than about 12 inches (in.) to facilitate sunlight penetration, such as in the range of about 5 in. to about 12 in., encompassing any value and subset therebetween. Accordingly, the size of the largest cultivation pond is preferably selected to hold a volume of the algae slurry sufficient to seed each of the at least two smaller cultivation ponds at desired, identical depths (e.g., each smaller pond is seeded with the algae slurry at a depth of 5 in., or 8 in., or 12 in. and the like). If the largest cultivation pond does not have enough freeboard (e.g., volumetric) space to accommodate the necessary volume of algae slurry to be prepared, a separate tank or reservoir may be utilized in concert with the largest cultivation pond. That is, any suitable container may be used to cultivate the initial (“mother”) algae slurry described in accordance with the methods provided herein. It is noted that greater than three cultivation ponds and cultivation ponds that are larger than 4000 m² or smaller than 2 m² may be utilized according to the embodiments described herein, without departing from the scope of the present disclosure.

More specifically, according to one or more embodiments, an algae culture “seed stock” is initially prepared. Algal sources for the preparing the seed stock include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

The prepared seed stock and algae nutrient media are added to the largest selected cultivation pond comprising water, thereby forming an algae slurry in the largest cultivation pond. The order in which the seed stock and the algae nutrient media are added to the water in the largest cultivation pond is not critical and either may be added before the other or they may be added simultaneously, without departing from the scope of the present disclosure. For example, the seed stock may initially be mixed with only water in the largest cultivation pond (e.g., using one or more paddlewheels), the mixture circulated for a time sufficient to achieve a homogeneous or near-homogeneous mixture thereof throughout the pond, and thereafter, the algae nutrient media may be added and further mixed. Alternatively, the order may be switched (i.e., algae nutrient media added first and thereafter the seed stock is added after mixing). Still alternatively, the seed stock and algae nutrient media may be added simultaneously to the water and the mixture circulated for a time sufficient to achieve a homogeneous or near-homogeneous mixture thereof throughout the pond.

The water in the largest cultivation pond may be from any water source including, but not limited to, fresh water, brackish water, seawater, wastewater (treated or untreated), synthetic seawater, and any combination thereof. The wastewater may derive, for example, from previously cultivated algae slurries after separation and removal of the algae components. The synthetic seawater may, for example, be prepared by dissolving salts into fresh water. The algae nutrient media may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

According to one or more embodiments described herein, the algae (i.e., from the seed stock) may be initially in a concentration in the algae slurry in the largest selected cultivation pond in an amount in the range of about 0.1 grams per liter (g/L) to about 0.3 g/L, encompassing any value and subset therebetween.

The homogeneous or near-homogeneous algae slurry mixture residing in the largest cultivation pond represents the entirety of algae slurry used to evaluate the impact of scaling and may be referred to as the “mother” slurry. An appropriate volume of the algae slurry is transferred from the largest cultivation pond into each of the at least two (or more) smaller cultivation ponds to achieve equivalent depths in each such smaller cultivation pond (e.g., between 5-12 in.). The smaller cultivation ponds comprise no additional contents.

Simultaneous cultivation of the algae slurry is performed in the at least two smaller cultivation ponds at an equivalent bulk liquid flow velocity. The bulk liquid flow velocities may be in the range of about 0.1 meters per second (m/s) to about 0.5 m/s, encompassing any value and subset therebetween. In some examples, the bulk liquid flow velocities of the at least two smaller cultivation ponds is about 0.3 m/s.

The algae slurries are cultivated in the circulation in the at least two smaller cultivation ponds for an equivalent predetermined period of time. For example, the cultivation may be between about 1 week to about 3 weeks, encompassing any value and subset therebetween. In some examples, the cultivation is about 1 week. In some embodiments, the predetermined period of time for the cultivation is based on a predetermined final concentration of algal biomass (i.e., cultivated algae cells) in the algae slurry. For example, the predetermined period of time may be the time necessary for the algal biomass to reach a final concentration in the algae slurry in the range of about 1.0 g/L to about 1.5 g/L, encompassing any value and subset therebetween.

Accordingly, each of the at least two smaller cultivation ponds (of different sizes) comprise an identical algae slurry having identical initial biological process variables, such as those described above (e.g., salinity, dissolved oxygen, nutrient media content, algae density, algae stress, dissolved carbon dioxide, initial pH, and the like). Further, each of the at least two smaller cultivation ponds are simultaneously operated to cultivate the identical algae slurry under identical bulk liquid flow velocities and during identical periods of time, further controlling wholly for weather process variables, such as those described above (e.g., ambient temperature, humidity, wind speed and shear, PAR exposure, and the like). Therefore, the methods described herein control, or at least control at the time of initial pond cultivation, variables that are not attributable to scaling factors (e.g., those associated with the size of a cultivation pond, the hydrodynamics of the cultivation pond, and the like). As such, the impact of scaling can be assessed based on a comparison of the quality and quantity of the resultant algal biomass cultivated in each of the different sized two or more cultivation ponds. That is, a dataset pertaining to each algal biomass may be obtained where differences between the dataset are attributable wholly or in large part to the influence of scaling, thereby providing important information pertinent to designing a large-scale cultivation system.

The quality and quantity of the resultant algal biomass from each sized cultivation pond may be evaluated by various observational and laboratory techniques. For example, evaluation of the algal biomass may include, but is not limited to, ash-free dry weight, total organic carbon, density, lipid concentration (e.g., total lipids as fatty acid esters), dissolved oxygen, salinity, elemental analysis, and the like, and any combination thereof. By characterizing the quality and quantity of the algal biomass in each of the at least two different sized cultivation ponds, a scale-up design may be informed, including whether a particular sized cultivation pond is capable of achieving desired production rates (e.g., algal biomass concentration over a particular period of time). In some embodiments, a predictive model or other evaluation process may be designed based on the data obtained from evaluating the algal biomass, weather data (e.g., light exposure, wind, temperature, and the like during the cultivation time), as well as hydrodynamic flow pattern of the cultivation ponds to inform scale-up design.

Referring to the FIGURE, depicted is a schematic representation of an example method 100 described herein for scale-up evaluation of algal biomass cultivation, according to one or more embodiments. Four cultivation ponds 102, 104, 106, and 108 are shown, each having a different volumetric size. Cultivation pond 102 has the largest volumetric size, cultivation pond 104 has the next largest volumetric size, cultivation pond 106 has the next largest volumetric size, and cultivation pond 108 has the smallest volumetric size.

In accordance with the embodiments of the present disclosure, an algae slurry is initially mixed in the largest cultivation pond 102, the algae slurry comprising at least water, algae cells (e.g., from a seed stock), and algae nutrient media. The algae slurry is mixed in pond 102 until a homogeneous or near homogeneous mixture is achieved. Thereafter, the algae slurry mixture is transferred to each of ponds 104, 106, and 108 at an equivalent desired depth (e.g., no greater than 12 ft.). No additional contents are added to ponds 104, 106, and 108 in order to control various process variables, as described herein, and isolate the impact of scaling. The algae slurries in ponds 104, 106, and 108 are allowed to cultivate for an equivalent predetermined period of time and an equivalent bulk liquid flow velocity. Thereafter, one or more qualities and the quantity of the resultant algal biomass are evaluated to understand the impact of scaling and to design a scale-up operation.

While four cultivation ponds are shown in the FIGURE, greater than four or at least three may be utilized according to the embodiments described herein to evaluate the impact of scaling of algae cultivation, provided that the ponds are of different sizes or geometric ratios and the largest can be used to mix an algae slurry to be transferred to the smaller cultivation ponds.

### Embodiments Listing

The present disclosure provides, among others, the following embodiments, each of which may be considered as optionally including any alternate embodiments.

Clause 1. A method comprising: mixing an algae water slurry in a first cultivation pond; transferring the algae water slurry into at least two additional cultivation ponds, the at least two additional cultivation ponds having different volumetric sizes and each having a volumetric size less than the first cultivation pond; cultivating the algae water slurry in the at least two additional cultivation ponds for an equivalent predetermined period of time, and thereby growing an algal biomass; and evaluating a quality and quantity of the algal biomass to determine influence of scaling.

Clause 2. The method of Clause 1, further comprising determining an optimal cultivation volumetric pond size based on the evaluating.

Clause 3. The method of Clause 2, wherein the optimal volumetric cultivation pond size is based on an algal biomass production rate.

Clause 4. The method of any of the preceding Clauses, wherein the quality of the algal biomass is selected from the group consisting of ash-free dry weight, total organic carbon, density, lipid concentration, dissolved oxygen, salinity, elemental analysis, and any combination thereof.

Clause 5. The method of any of the preceding Clauses, wherein the first cultivation pond and the at least two additional cultivation ponds have an area in the range of about 2 square meter to about 10,000 square meters.

Clause 6. The method of any of the preceding Clauses, wherein the equivalent predetermined period of time is in the range of about 1 week to about 3 weeks.

Clause 7. The method of any of the preceding Clauses, wherein the predetermined period of time is based on a final concentration of the algal biomass in the cultivated algae water slurry in the range of about 1.0 grams per liter to about 1.5 grams per liter.

Clause 8. The method of any of Clauses 1 to 6, wherein the predetermined period of time is based on a final concentration of the algal biomass in the cultivated algae water slurry in the range of about 1.0 grams per liter to about 1.5 grams per liter.

Clause 9. The method of any of the preceding Clauses, further comprising designing a scale-up operation based on at least the evaluating.

Clause 10. The method of Clause 9, further comprising designing the scale-up operation additionally based on one or more of weather data and hydrodynamic flow pattern of the at least two additional cultivation ponds.

Clause 11. The method of any of the preceding Clauses, further comprising cultivating the algae water slurry in the at least two additional cultivation ponds at an equivalent bulk liquid flow velocity.

Clause 12. The method of Clause 11, wherein the equivalent bulk liquid flow velocity is in the range of about 0.5 meters per second to about 0.5 meters per second.

Clause 13. The method of Clause 11, wherein the equivalent bulk liquid flow velocity is about 0.3 meters per second.

Clause 14. The method of any of the preceding Clauses, further comprising cultivating the algae water slurry in the at least two additional cultivation ponds at an equivalent depth.

Clause 15. The method of Clause 14, wherein the depth is in the range of 5 inches to 12 inches.

Clause 16. The method of any of the preceding Clauses, where in the algae slurry comprises water, algae cells, and algae nutrient media.

Clause 17. The method of Clause 16, wherein the algae cells are one or more of unicellular and multicellular.

Clause 18. The method of Clause 16, wherein the algae nutrient media comprises at least nitrogen and phosphorous.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The invention claimed is:

1. A method comprising:
mixing an algae water slurry in a first cultivation pond;
transferring the algae water slurry into at least two additional cultivation ponds, the at least two additional cultivation ponds having different volumetric sizes and each having a volumetric size less than the first cultivation pond;
cultivating the algae water slurry in the at least two additional cultivation ponds for an equivalent predetermined period of time, and thereby growing an algal biomass; and
evaluating a quality and quantity of the algal biomass to determine influence of scaling.

2. The method of claim 1, further comprising determining an optimal cultivation volumetric pond size based on the evaluating.

3. The method of claim 2, wherein the optimal volumetric cultivation pond size is based on an algal biomass production rate.

4. The method of claim 1, wherein the quality of the algal biomass is selected from the group consisting of ash-free dry weight, total organic carbon, density, lipid concentration, dissolved oxygen, salinity, elemental analysis, and any combination thereof.

5. The method of claim 1, wherein the first cultivation pond and the at least two additional cultivation ponds have an area in the range of about 2 square meter to about 10,000 square meters.

6. The method of claim 1, wherein the equivalent predetermined period of time is in the range of about 1 week to about 3 weeks.

7. The method of claim 1, wherein the predetermined period of time is based on a final concentration of the algal biomass in the cultivated algae water slurry in the range of about 1.0 grams per liter to about 1.5 grams per liter.

8. The method of claim 1, further comprising designing a scale-up operation based on at least the evaluating.

9. The method of claim 8, further comprising designing the scale-up operation additionally based on one or more of weather data and hydrodynamic flow pattern of the at least two additional cultivation ponds.

10. The method of claim 1, further comprising cultivating the algae water slurry in the at least two additional cultivation ponds at an equivalent bulk liquid flow velocity.

11. The method of claim 10, wherein the equivalent bulk liquid flow velocity is in the range of about 0.1 meters per second to about 0.5 meters per second.

12. The method of claim 10, wherein the equivalent bulk liquid flow velocity is about 0.3 meters per second.

13. The method of claim 1, further comprising cultivating the algae water slurry in the at least two additional cultivation ponds at an equivalent depth.

14. The method of claim 13, wherein the depth is in the range of 5 inches to 12 inches.

15. The method of claim 1, where in the algae slurry comprises water, algae cells, and algae nutrient media.

16. The method of claim 15, wherein the algae cells are one or more of unicellular and multicellular.

17. The method of claim 15, wherein the algae nutrient media comprises at least nitrogen and phosphorous.
